(19) United States
(12) Patent Application Publication
(10) Pub. No.: US 2008/0249528 A1
(43) Pub. Date: Oct. 9, 2008

(54) Rotating Interspinous Device

(76) Inventor: Charles Khalife, Amiens (FR)

Correspondence Address: GREENBLUM & BERNSTEIN, P.L.C., 1950 ROLAND CLARKE PLACE, RESTON, VA, 20191, US

(21) Appl. No.: 11/996,219

(22) PCT Filed: Jul. 19, 2006
(86) PCT No.: PCT/FR2006/001773

§ 371 Date: Jun. 9, 2008

(30) Foreign Application Priority Data
Jul. 21, 2005 (FR) 0507747

(51) Int. Cl. A61B 17/58; A61F 2/44

(52) U.S. Cl. 606/90; 623/17.16

(57) ABSTRACT

The invention concerns an implant which consists in a device used between the spinous processes to produce a distraction and a decompression. It consists of two separate parts, one female (1) and the other male (2), each fixed to a spinous process through indentations (3) adapted, and socketed into each other to form a single articulated part. The socketed parts, male and female, are spherical (6, 7) or cylindrical, to enable a rotating or multidirectional movement of the device. The spherical (6) and cylindrical orifices of the parts enabling an additional translational movement. An assembling screw (19) may be added to the device, to prevent a possible dismantling of the two parts (1, 2), while maintaining the multidirectional movements.

The present invention relates to an implant adapted to be positioned in the area of the spine, and more particularly between the spinous processes, in order to restore the height of the intervertebral disc space and to decompress the neural elements. The implant is generally used alone in the case of symptomatic compression, or at the end of the assembly in conjunction with a spinal osteosynthesis, to decrease the stresses to which the region adjacent to the fixed region is subjected.

The invention relates more particularly to improvements to this type of implants. Several models of interspinous implants are currently available, and in spite of the improvements developed by manufacturers, none of these implants is entirely satisfactory.

Generally, interspinous implants are unitary elements and do not respect the physiological movements of the spine three-dimensional rotation, which can cause a conflict between the spinous processes and the implants. Other models have several parts in their assembly, thus running the risk of disassembly or wear and tear debris.

The implant according to the invention makes it possible to efficiently overcome the two problems related to the rotation and to the risk of disassembly or wear and tear debris.

The implant according to the invention is comprised of two distinct parts adapted to be movable with respect to one another and to be articulated to form a single movable part. A fastening screw can be added to the device to prevent separation of the two parts. Each of the two parts is fixed to a spinous process on one side, due to a matching notch, and embedded in the other part on the other side, to form a single, articulated part having three-dimensional mobility.

According to a characteristic of the invention, the portions of the anchoring parts adapted to be embedded, one female and the other male, can be cylindrical to enable rotational movements, or spherical to ensure multidirectional movements.

According to another characteristic of the invention, the embedding opening of the cylindrical or spherical female portion can have an oblong, straight or circular arc shape to allow an additional translational movement.

According to an advantageous arrangement of the invention, the male and female embedding portions can have various heights representing several sizes for the device in order to adapt to the interspinous space.

According to another advantageous characteristic of the invention, a fastening screw can be added to connect the two parts to one another and to prevent their separation while maintaining the possibility of three-dimensional movement.

The two parts of the device can be manufactured with various types of biocompatible materials that can withstand friction, such as titanium, stainless steel, for example, or any other suitable material.

Other characteristics and advantages of the invention will become apparent from the description that follows, with reference to the annexed drawings which are given only by way of non-limiting examples.

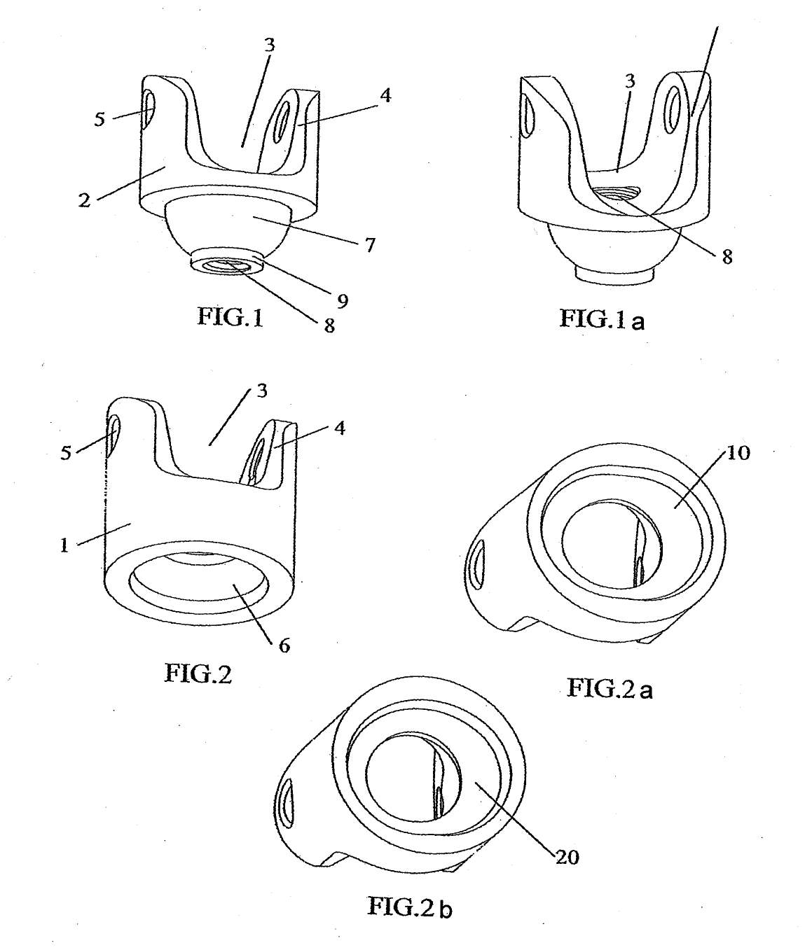

FIGS. 1 to 9 show the various parts of the system according to the invention.

FIGS. 1, 1a, 2, 2a, 2b, 5, and 6, show the various parts of a first embodiment of the system according to the invention, according to which the cooperation between the two anchoring parts is spherical in order to provide multidirectional mobility between the two anchoring parts,

FIG. 1 is a perspective bottom view of the underside of the spherical male anchoring part.

FIG. 1a is a perspective top view of the spherical male anchoring part.

FIG. 2 is a perspective bottom view of the spherical female anchoring part.

FIG. 2a is a perspective bottom view of the spherical female anchoring part with a straight oblong slit.

FIG. 2b is a perspective bottom view of the spherical female anchoring part with a circular arc oblong slit.

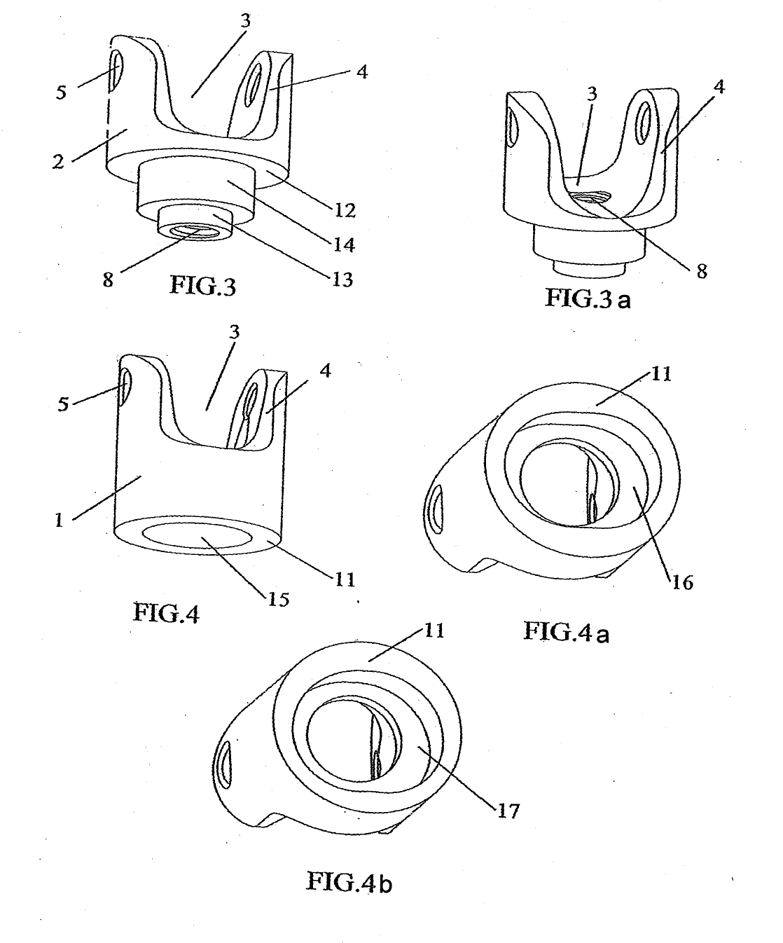

FIGS. 3, 3a, 4, 4a, 4b, 7, 8, and 9, show the various parts of a second embodiment of the system according to the invention, according to which the cooperation between the two anchoring parts is cylindrical in order to provide rotational mobility between the two anchoring parts.

FIG. 3 is a perspective bottom view of the cylindrical male anchoring part.

FIG. 3a is a perspective top view of the cylindrical male anchoring part.

FIG. 4 is a perspective bottom view of the cylindrical female anchoring part.

FIG. 4a is a perspective bottom view of the cylindrical female anchoring part, with a straight oblong slit.

FIG. 4b is a perspective bottom view of the cylindrical female anchoring part with a circular arc oblong slit.

FIG. 5 is a perspective, exploded view of the two anchoring parts with the fastening screw.

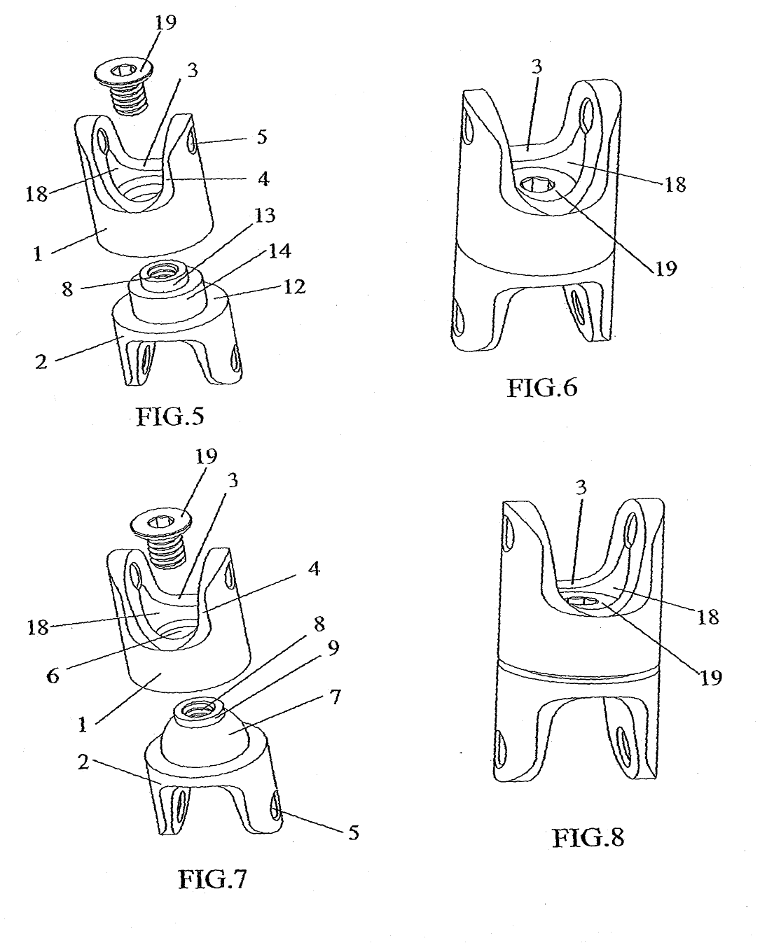

FIG. 6 is a perspective view of the assembly of the two cylindrical anchoring parts.

FIG. 7 is a view of the two spherical anchoring parts with the fastening screw.

FIG. 8 is a view of the assembly of the two spherical anchoring parts.

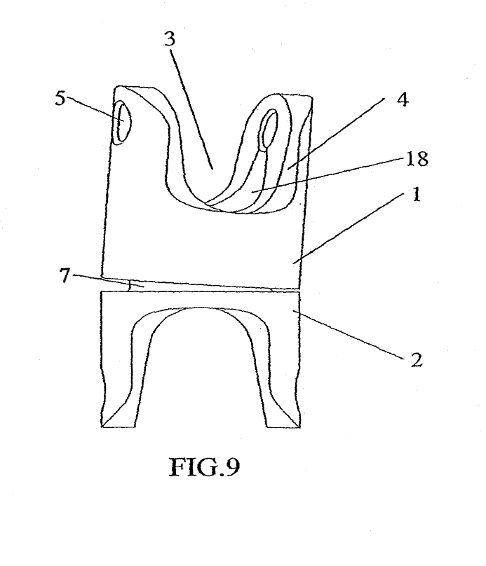

FIG. 9 is a view of the assembly, with inclination, of the two spherical anchoring parts.

The annexed drawings illustrate the invention.

The operating mechanism of the rotatory interspinous device is described hereinafter, with reference to FIGS. 1 to 9.

For a clearer description of the invention, the two anchoring parts of the device, adapted to be embedded, will be referred to as female and male anchoring parts. These female and male anchoring parts can be spherical or cylindrical.

The implant of the invention is characterized in that it includes two anchoring parts, one female (1) and the other male (2), adapted to anchor themselves to the spinous processes on one side, and to cooperate with one another by becoming embedded in one another on the other side by cooperation of forms, a spherical or cylindrical cooperation of forms, to form a single, articulated part that is capable of carrying out rotational movements for one of the embodiments, or even multidirectional movements for the other embodiment.

According to a characteristic of the invention, the two spherical or cylindrical female (1) and male (2) anchoring parts have notches (3) enabling them to fix themselves to the spinous processes.

According to an advantageous arrangement, the notches (3) are limited by vertical walls (4) having an optimal height to allow positioning a second device at the level of the adjacent region.

According to another advantageous arrangement, the vertical walls (4) are provided with openings (5) to make it possible to insert an anchoring system that will fix the female (1) and male (2) anchoring parts to the spinous processes. The anchoring system can be rigid, such as a screw or a pin, or flexible, such as a suture or a synthetic ligament.

According to a characteristic of the invention, the anchoring female part (1) is provided, on the side opposite the notch (3), with a cooperating opening or housing (6, 13), which is spherical (6) for one of the embodiments, or cylindrical (15) for the other embodiment, and is adapted to receive the sphere (7) or cylinder (14) of the corresponding male anchoring part (2).

According to another characteristic of the invention, the male anchoring part (2) has, on the side opposite the notch (3), a spherical (7) or cylindrical (14) portion adapted to be embedded in the spherical (6) or cylindrical (15) opening of the female part (2) to connect the two parts (1), (2) of the device, and to ensure mobility therebetween, and, in particular, rotation and/or multidirectional movement.

According to an advantageous arrangement of the invention, the spherical (7) or cylindrical (14) male portion is provided with a threading (8) on the side opposite the notch (3), which can open out in the latter to receive the fastening screw (19) that prevents separation of the two assembled parts. The fastening screw (19) is inserted through an opening (18) made at the bottom of the notch (3) of the female anchoring part (1).

According to another advantageous arrangement of the invention, the distance between the notches (3) of the two anchoring parts (1), (2) mounted in one another, constitutes the height defining the size of the device (FIG. 6, FIG. 8).

According to another characteristic of the invention, the cylindrical male anchoring part (2) has, at the base of the cylindrical portion (14), a support flange (12) that serves as an abutment for the support wall (11) of the cylindrical opening (15), to define the desired height of the device, measured between the two notches (3). For the spherical anchoring parts, the height is ensured by the abutment between the spherical female (6) and male (7) portions.

According to an advantageous arrangement of the invention, the distance between the notch (3) of the cylindrical male anchoring part (2) and the flange (12), on which the wall (11) of the cylindrical opening (15) rests, can have several heights to increase the height of the assembled device (FIG. 6) and to adapt to the anatomical interspinous space to be filled. For the spherical male anchoring part (2), the distance between the notch (3) and the spherical dome (7) can have several heights to define several sizes for the device.

According to another advantageous arrangement of the invention, the height of the assembled device (FIGS. 6 and 8) can also be increased by the increase in the height of the spherical or cylindrical female anchoring part (1) beneath the notch (3).

According to an advantageous arrangement of the invention, the spherical (6) or cylindrical (15) opening of the female anchoring part (1) can have a straight oblong (10, 16) or circular arc (20, 17) shape to ensure a translational movement that completes the rotational movement.

According to an advantageous arrangement of the invention, the spherical (7) and cylindrical (14) portions of the male anchoring parts (2) can end in a small cylindrical segment (9, 13) adapted to be used as an abutment limiting the amplitude of the multidirectional movements.

1. Device for spinal surgery, of the implant type, adapted to be positioned in distraction in the interspinous space, characterized in that it comprises two distinct parts, namely two anchoring parts, one female (1) and the other male (2), each fixed to a spinous process by means of a notch (3), said anchoring parts (1, 2) being connected in order to be articulated with respect to one another due to two cylindrical or spherical portions, the one female (6, 15) and the other male (7, 14), to ensure a rotational or multidirectional movement.

2. Interspinous device according to claim 1, characterized in that the female (1) and male (2) anchoring parts have matching notches (3) to fix them to the spinous process.

3. Interspinous device according to claim 1, characterized in that the cylindrical female anchoring part (1) has, on the side opposite the notch (3), a cylindrical opening (15) adapted to receive the cylindrical male portion (14).

4. Device according to claim 1, characterized in that the cylindrical opening (15) of the cylindrical female anchoring part (1) can have a straight oblong (16) or circular arc (17) shape to allow an additional translational movement.

5. Device according to claim 1, characterized in that the cylindrical male anchoring part (2) has, on the side opposite the notch (3), a cylindrical portion (14) adapted to be embedded in the cylindrical opening (15) to form a rotatory system, with possibility of translation, between the two cylindrical parts of the device.

6. Device according to claim 1, characterized in that the spherical female anchoring part (1) has, on the side opposite to the notch (3), a spherical opening (6) adapted to receive the spherical male portion (7).

7. Device according to claim 1, characterized in that the spherical opening (6) of the spherical female anchoring part (1) can have a straight oblong (10) or circular arc (20) shape to allow an additional translational movement.

8. Device according to claim 1, characterized in that the spherical male anchoring part (2) has, on the side opposite the notch (3), a spherical portion (7) adapted to be embedded in the spherical opening (6) to form a multidirectional rotatory system, with possibility of translation, between the two spherical parts of the device.

9. Device according to claim 1, characterized in that the cylindrical or spherical female (1) or male (2) anchoring parts have several heights to form a device of various sizes intended to adapt to the anatomical needs

10. Device according to claim 1, characterized in that the female (1) and male (2) anchoring parts are connected, after being embedded, by a fastening screw (19) that prevents a possible separation, while maintaining the relative movements between the two anchoring parts (1, 2).
